(19) United States
(12) Patent Application Publication
(10) Pub. No.: US 2024/0059770 A1
(43) Pub. Date: Feb. 22, 2024

(54) OXYNTOMODULIN BINDING MOLECULES, AND USES THEREOF

(71) Applicant: Eli Lilly and Company, Indianapolis, IN (US)

(72) Inventors: Robert John KONRAD, Carmel, IN (US); Wenyu MING, Carmel, IN (US); Robert William SIEGEL, II, Fountaintown, IN (US); Tara Suzanne UMBERGER, Frankton, IN (US)

(21) Appl. No.: 18/259,892

(22) PCT Filed: Jan. 19, 2022
(86) PCT No.: PCT/US2022/012898

§ 371 Date: Jun. 29, 2023

Related U.S. Application Data
(60) Provisional application No. 63/139,428, filed on Jan. 20, 2021.

(51) Int. Cl. C07K 16/26; C12N 15/63; G01N 33/74

(52) U.S. Cl. CPC C07K 16/26; C12N 15/63; G01N 33/74; C07K 2317/14; C07K 2317/565; G01N 2800/50

(57) ABSTRACT

Provided herein are novel N- and C-terminal oxyntomodulin polypeptide binding molecules, and uses thereof, including a method for quantitating oxyntomodulin.

Oxyntomodulin is a proglucagon-derived peptide agonist of both the glucagon-like peptide-1 (GLP-1) and glucagon receptors, and is a key regulator of gastric acid secretion and energy expenditure. Oxyntomodulin is a potential target for therapeutic intervention (Pocai A, Mol. Metab. 2014; 3:241-51), and has been proposed as a potential biomarker for predicting if a subject is at risk of developing type 2 diabetes after acute pancreatitis (Bharmal, S H, et al., Clin. Transl. Gastroenterology 2020; 11: page e00132, doi: 10.14309/ctg.00000000 00000132).

Human proglucagon is cleaved into a number of different peptides in a tissue-specific manner (Holst J J, et al., Peptides 2018; 100: 48-53), and sequence similarities between proglucagon cleavage peptides have made the quantitation of endogenous levels of oxyntomodulin difficult. Historically, polyclonal antibodies directed to specific regions of glucagon (33-61) or the C-terminal octapeptide (62-69) have been used to quantitate oxyntomodulin-like immunoreactive (OLI) components in various tissues and plasma. Initially, a two-step subtractive radioimmunoassay was used to estimate OLI after the specific subtraction of pancreatic glucagon levels (Kervran A, et al., Endocrinology 1987; 121:704-1). Subsequent assays used polyclonal antibodies directed to the C-terminal octapeptide to directly measure OLI components (Blache P, et al., Anal Biochem 1988; 173:151-9; Collie N L, et al., Proc Natl Acad Sci USA 1994; 91:9362-6; Le Quellec A, et al., J. Clin. Endocrinol. Metab 1992; 74:1405), and assays based on that approach are now commercially available.

Nonetheless, estimates of endogenous OLI in human plasma vary widely from 60 to 5000 ng/L in the fasted state (Laferrere B, et al., J. Clin. Endocrinol. Metab. 2010; 95:4072-6; Le Quellec A, et al., J. Clin. Endocrinol. Metab 1992; 74:1405-9). Furthermore, the proportion of OLI represented by glicentin confounds accurate oxyntomodulin quantification, and obscures the physiological role(s) of oxyntomodulin (Bak M J, et al., Eur. J. Endocrinol. 2014; 170:529-3). Oxyntomodulin can be distinguished from glicentin using HPLC or LC-MS, but those approaches lack sufficient analytical sensitivity to quantitate endogenous levels of oxyntomodulin in plasma (Lee A Y, et al., Clin. Chem. 2015; 62: 227-235). A sandwich assay has been reported (Albrechtsen, N J, et al., EBioMedicine 2016; 7: 112-120), but it lacks specificity, because it is characterized by 10% cross-reactivity with glicentin (Holst J J, el al., Peptides 2018, 100: 48-53).

Thus, decades after the discovery of oxyntomodulin, the research community has been unable to selectively and reliably measure endogenous levels of oxyntomodulin in human plasma samples, and there remains a need for an assay that is not only selective and sensitive, but that is also amendable to high throughput application.

A sandwich immunoassay is an assay that utilizes two antibodies that bind to different sites on an antigen of interest. However, to implement an oxyntomodulin sandwich immunoassay, antibodies are required that (a) bind distinct epitopes of respective proglucagon fragments, and (b) detect oxyntomodulin only when used together in the assay. The present invention provides antibodies and methods that facilitate a sandwich assay method for quantitating oxyntomodulin and a predictive method for determining if a subject is at risk of developing diabetes.

The present invention provides a polypeptide molecule that binds the N-terminal region of each of human oxyntomodulin (SEQ ID NO: 25) and human glucagon (SEQ ID NO: 30), wherein the polypeptide molecule comprises the complementarity determining regions (CDRs) set forth in SEQ ID NOS: 1-6. In one embodiment, the polypeptide molecule is an antibody. In another embodiment, the polypeptide molecule is an antibody fragment, wherein the antibody fragment binds the N-terminal region of each of human oxyntomodulin (SEQ ID NO: 25) and human glucagon (SEQ ID NO: 30) and comprises SEQ ID NOS: 1-6. In another embodiment, the antibody fragment is an scFv. In another embodiment, the antibody fragment is a Fab.

In another embodiment, the polypeptide molecule is an antibody comprising a heavy chain variable region (HCVR or VH) comprising SEQ ID NO: 7 and a light chain variable region (LCVR or VL) comprising SEQ ID NO: 8. In another embodiment, the polypeptide molecule is an antibody comprising a heavy chain comprising SEQ ID NO: 9 and a light chain comprising SEQ ID NO: 10. In another embodiment, the polypeptide molecule is an antibody comprising a heavy chain consisting of a SEQ ID NO: 9 and a light chain consisting of SEQ ID NO: 10. The present invention also provides a composition comprising the polypeptide molecule.

The present invention also provides a nucleic acid molecule comprising one or both of a first nucleic acid sequence encoding SEQ ID NO: 9 and a second nucleic acid sequence encoding SEQ ID NO: 10. In another embodiment, the first nucleic acid sequence encoding SEQ ID NO: 9 comprises the nucleic acid of SEQ ID NO: 11, and the second nucleic acid sequence encoding SEQ ID NO: 10 comprises the nucleic acid of SEQ ID NO: 12. The present invention also provides a vector comprising one or both of a first nucleic acid sequence encoding SEQ ID NO: 9 and a second nucleic acid sequence encoding SEQ ID NO: 10. The present invention also provides a composition comprising the vector. The present invention also provides a cell comprising the vector. In one embodiment, the cell is a mammalian cell. The present invention also provides a process of producing a polypeptide molecule, comprising culturing the cell under conditions such that the polypeptide molecule is expressed, and recovering the expressed polypeptide molecule from the culture medium. The present invention also provides a polypeptide molecule produced by the process.

The present invention also provides a polypeptide molecule that binds the C-terminal region of each of human oxyntomodulin (SEQ ID NO: 25) and human glicentin (SEQ ID NO: 28), wherein the polypeptide molecule comprises the complementarity determining regions set forth in SEQ ID NOS: 13-18. In one embodiment, the polypeptide molecule is an antibody. In another embodiment, the polypeptide molecule is a fragment of the antibody, wherein the fragment binds the C-terminal region of each of human oxyntomodulin (SEQ ID NO: 25) and human glicentin (SEQ ID NO: 28). In another embodiment, the fragment of the polypeptide molecule is an scFv. In another embodiment, the fragment of the polypeptide molecule is a Fab.

In another embodiment, the polypeptide molecule is an antibody comprising a VH comprising SEQ ID NO: 19 and a VL comprising SEQ ID NO: 20. In another embodiment, the polypeptide molecule is an antibody comprising a heavy chain comprising SEQ ID NO: 21 and a light chain comprising SEQ ID NO: 22. In another embodiment, the polypeptide molecule is an antibody comprising a heavy chain consisting of a SEQ ID NO:21 and a light chain consisting of SEQ ID NO: 22. The present invention also provides a composition comprising the polypeptide molecule.

The present invention also provides a nucleic acid molecule comprising one or both of a first nucleic acid sequence encoding SEQ ID NO: 21 and a second nucleic acid sequence encoding SEQ ID NO: 22. In one embodiment, the first nucleic acid sequence encoding SEQ ID NO: 21 comprises the nucleic acid of SEQ ID NO: 23, and the second nucleic acid sequence encoding SEQ ID NO: 22 comprises the nucleic acid of SEQ ID NO: 24. The present invention also provides a vector comprising one or both of a first nucleic acid sequence encoding SEQ ID NO: 23 and a second nucleic acid sequence encoding SEQ ID NO: 24. The present invention also provides a composition comprising the vector. The present invention also provides a cell comprising the vector. In one embodiment, the cell is a mammalian cell. The present invention also provides a process of producing a polypeptide molecule, comprising culturing the cell under conditions such that the polypeptide molecule is expressed, and recovering the expressed polypeptide molecule from the culture medium. The present invention also provides a polypeptide molecule produced by the process.

The present invention also provides a composition comprising (a) a first polypeptide molecule that binds the N-terminal region of each of human oxyntomodulin (SEQ ID NO: 25) and human glucagon (SEQ ID NO: 30), wherein the first polypeptide molecule comprises SEQ ID NOS: 1-6, and (b) a second polypeptide molecule that binds the C-terminal region of each of human oxyntomodulin (SEQ ID NO: 25) and human glicentin (SEQ ID NO: 28), wherein second the polypeptide molecule comprises SEQ ID NOS: 13-18.

The present invention also provides a sandwich assay method for determining the amount of human oxyntomodulin (SEQ ID NO: 25) in a liquid sample, comprising: (a) contacting a liquid sample comprising human oxyntomodulin with a first polypeptide molecule that binds the C-terminal region of each of human oxyntomodulin (SEQ ID NO: 25) and human glicentin (SEQ ID NO: 28), wherein the first polypeptide molecule comprises SEQ ID NOS: 13-18, thereby forming a first polypeptide molecule-human oxyntomodulin complex; (b) contacting a second polypeptide molecule that binds the N-terminal region of each of human oxyntomodulin (SEQ ID NO: 25) and human glucagon (SEQ ID NO: 30), wherein the second polypeptide molecule comprises SEQ ID NOS: 1-6, thereby forming a first polypeptide molecule-human oxyntomodulin-second polypeptide molecule complex; and (c) quantitating the amount of oxyntomodulin in the first polypeptide molecule-human oxyntomodulin-second polypeptide molecule complex by comparison against a standard curve of known amounts of human oxyntomodulin (SEQ ID NO: 25).

In another embodiment of the methods of the invention, the first polypeptide molecule is an antibody and the second polypeptide molecule is an antibody. In another embodiment, the first polypeptide molecule is an antibody comprising a VH comprising SEQ ID NO: 19 and a VL comprising SEQ ID NO: 20, and the second polypeptide molecule is an antibody comprising a VH comprising SEQ ID NO: 7 and a VL comprising SEQ ID NO: 8. In another embodiment, the first polypeptide molecule is an antibody comprising a heavy chain comprising SEQ ID NO: 21 and comprising a light chain comprising SEQ ID NO: 22, and the second polypeptide molecule is an antibody comprising a heavy chain comprising SEQ ID NO: 9 and a light chain comprising SEQ ID NO: 10. In another embodiment, the first polypeptide molecule is an antibody comprising a heavy chain consisting of SEQ ID NO: 21 and comprising a light chain consisting of SEQ ID NO: 22, and the second polypeptide molecule is an antibody comprising a heavy chain consisting of SEQ ID NO: 9 and a light chain consisting of SEQ ID NO: 10.

The present invention also provides a method of predicting if a subject is at risk of developing type 2 diabetes, comprising determining the centration of oxyntomodulin in a serum sample or a plasma sample from a subject, comprising: (a) contacting a serum sample or a plasma sample comprising human oxyntomodulin with a first polypeptide molecule that binds the C-terminal of each of human oxyntomodulin (SEQ ID NO: 25) and human glicentin (SEQ ID NO: 28), wherein the first polypeptide molecule comprises SEQ ID NOS: 13-18, thereby forming a first polypeptide molecule-human oxyntomodulin complex; (b) contacting a second polypeptide molecule that binds the N-terminal region of each of human oxyntomodulin (SEQ ID NO: 25) and human glucagon (SEQ ID NO: 30), wherein the second polypeptide molecule comprises SEQ ID NOS: 1-6, thereby forming a first polypeptide molecule-human oxyntomodulin-second polypeptide molecule complex; and (c) quantitating the amount of oxyntomodulin in the first polypeptide molecule-human oxyntomodulin-second polypeptide molecule complex by comparison against a standard curve of known amounts of human oxyntomodulin (SEQ ID NO: 25). In one embodiment of the method, the subject has been diagnosed with acute pancreatitis.

The present invention also provides the use of a first polypeptide molecule that binds the C-terminal octapeptide present in each of human oxyntomodulin (SEQ ID NO: 25) and human glicentin (SEQ ID NO: 28), and a second polypeptide molecule that binds the N-terminal region of each of human oxyntomodulin (SEQ ID NO: 25) and human glucagon (SEQ ID NO: 30), in determining the concentration of oxyntomodulin in a liquid sample.

In one embodiment of the methods of the invention, the liquid sample is serum. In another embodiment, the liquid sample is plasma.

In the methods of the invention, the first polypeptide molecule can be attached to a solid support. In one embodiment, the solid surface is a plate. In another embodiment, the solid surface is a plurality of beads. Such solid supports include, e.g., without limitation, glass, cellulose, plastic, poly-acrylamide, nylon, polystyrene, polyvinyl chloride, or polypropylene.

In another embodiment of the methods of the invention, the first polypeptide molecule is attached directly to the solid surface. In another embodiment, the first polypeptide molecule is indirectly attached to the solid surface with an attachment agent. In one embodiment, the attachment agent is streptavidin, neutravidin or avidin that is coated on the solid surface, and the polypeptide molecule is biotinylated.

In another embodiment of the methods of the invention, prior to contacting the liquid sample with the first polypeptide molecule, the solid surface is contacted with a blocking solution. In another embodiment, after contacting the liquid sample with the first polypeptide molecule, the first polypeptide molecule-human oxyntomodulin complex is contacted with a wash solution, thereby removing uncomplexed human oxyntomodulin.

In another embodiment of the methods of the invention, the first polypeptide molecule-human oxyntomodulin-second polypeptide molecule complex is contacted with a wash solution, thereby removing uncomplexed second polypeptide molecule.

In another embodiment of the method of determining the amount of human oxyntomodulin (SEQ ID NO: 25) in a liquid sample, e.g., plasma, the method comprises:
(a) contacting a solid surface, to which an antibody comprising a heavy chain comprising SEQ ID NO: 21 and a light chain comprising SEQ ID NO: 22 is attached, with a blocking solution;
(b) contacting a liquid sample comprising human oxyntomodulin with a first antibody comprising a heavy chain comprising SEQ ID NO: 21 and a light chain comprising SEQ ID NO: 22 attached to a solid surface, thereby forming a first antibody-human oxyntomodulin complex;
(c) contacting the solid surface with a wash solution, thereby removing uncomplexed human oxyntomodulin;
(d) contacting the solid surface with a second antibody comprising a heavy chain comprising SEQ ID NO: 9 and a light chain comprising SEQ ID NO: 10, thereby forming a first antibody-human oxyntomodulin-second antibody complex;
(e) contacting the solid surface with a wash solution, thereby removing uncomplexed second antibody; and
(f) quantitating the amount of oxyntomodulin in the first antibody-human oxyntomodulin-second antibody complex by comparison against a standard curve of known amounts of human oxyntomodulin (SEQ ID NO: 25).

In another embodiment of the predictive method of the invention, the method comprises:
(a) contacting a solid surface, to which an antibody comprising a heavy chain comprising SEQ ID NO: 21 and a light chain comprising SEQ ID NO: 22 is attached, with a blocking solution;
(b) contacting a liquid sample comprising human oxyntomodulin with a first antibody comprising a heavy chain comprising SEQ ID NO: 21 and a light chain comprising SEQ ID NO: 22 attached to a solid surface, thereby forming a first antibody-human oxyntomodulin complex;
(c) contacting the solid surface with a wash solution, thereby removing uncomplexed human oxyntomodulin;
(d) contacting the solid surface with a second antibody comprising a heavy chain comprising SEQ ID NO: 9 and a light chain comprising SEQ ID NO: 10, thereby forming a first antibody-human oxyntomodulin-second antibody complex;
(e) contacting the solid surface with a wash solution, thereby removing uncomplexed second antibody; and
(f) quantitating the amount of oxyntomodulin in the first antibody-human oxyntomodulin-second antibody complex by comparison against a standard curve of known amounts of human oxyntomodulin (SEQ ID NO: 25).

In another embodiment of the methods of the invention, the second polypeptide molecule is labeled. In another embodiment, quantification of the amount of second polypeptide is performed by quantitating the label. In another embodiment, the label is a radiolabel. In another embodiment, the radiolabel is ruthenium.

The polypeptide molecules, methods and uses of the invention facilitate quantifying oxyntomodulin that is selective and sensitive. In one embodiment, the method results in one or more of: a lower limit of oxyntomodulin quantitation (LLOQ) of 0.4 ng/L, no detection of glucagon, and/or less than 0.5% cross-reactivity with glicentin. An additional benefit of the method of the invention is that it facilitates quantitation of the pre- and post-meal levels of oxyntomodulin, with results that are highly correlated with results obtained using a conventional orthogonal IA-LC-MS assay.

Human proglucagon (SEQ ID NO: 36) contains 180 amino acid residues, and is encoded by the GCG gene (GCG-Pro-glucagon precursor-Homo sapiens (Human)-GCG gene & protein, unprot.org/uniprot/P01275 #sequences (2007)). This polypeptide precursor is differentially cleaved into a number of different peptides in a tissue-specific manner to generate a panel of peptides with different biological activities: a signal peptide (amino acid residues 1-20); human glicentin (amino acid residues 21-89); human glicentin-related pancreatic polypeptide (amino acid residues 21-50); human oxyntomodulin (amino acid residues 53-89); human glucagon (amino acid residues 53-81); human glucagon-like peptide 1 (GLP-1) (amino acid residues 92-128); human GLP-1 (7-37) (amino acid residues 98-128); human GLP-1 (7-36) (amino acid residues 98-127); and human glucagon-like peptide 2 (GLP-2) (amino acid residues 146-178). One outcome of the tissue-specific cleavage patterns from a common polypeptide precursor (SEQ ID 36) is the generation of biologically active peptides that have some sequence overlaps. For example, human oxyntomodulin (amino acid residues 53-89) contains all the sequences contained in human glucagon (amino acid residues 53-81) in addition to 8 amino acids that extend past amino acid residue 81. As another example, the sequences present in human oxyntomodulin (amino acid residues 53-89) and human glucagon (amino acid residues 53-81) are also contained in human glicentin (amino acid residues 21-89); however, human glicentin (amino acid residues 21-89) contains an additional 32 amino acids that precede residue 53 in both human oxyntomodulin (amino acid residues 53-89) and human glucagon (amino acid residues 53-81).

Sequences referred to herein are set forth in Table 1. The parenthetical numberings in certain sequence names in Table 1 refer to the respective amino acid residues in human proglucagon that includes the signal peptide sequence (residues 1-20). For example, in the term, “human oxyntomodulin (53-89),” the term “(53-89)” refers to amino acid residues 53-89 in human proglucagon. A deletion version of a human sequence in Table 1 is provided without the term “human.” For example, the term “oxyntomodulin (55-89)” refers to a human oxyntomodulin fragment with sequence that corresponds to amino acid residues 55-59 of human proglucagon, and that does not contain the first two N-terminal amino acid residues found in human oxyntomodulin.

TABLE 1
Sequences

| | | SEQ ID |
|---|---|---|
| | Name | NO: |
| Anti-Human | HCDR1 (amino acid (AA)) | 1 |
| Oxyntomodulin | HCDR2 (AA) | 2 |
| N-Terminal Antibody | HCDR3 (AA) | 3 |
| | LCDR1 (AA) | 4 |
| | LCDR2 (AA) | 5 |
| | LCDR3 (AA) | 6 |
| | Variable heavy region (VH) (AA) | 7 |
| | Variable light region (VL) (AA) | 8 |
| | Heavy chain (AA) | 9 |
| | Light chain (AA) | 10 |
| | Heavy Chain (DNA) | 11 |
| | Light Chain (DNA) | 12 |
| Anti-Human | HCDR1 (AA) | 13 |
| Oxyntomodulin | HCDR2 (AA) | 14 |
| C-Terminal Antibody | HCDR3 (AA) | 15 |
| | LCDR1 (AA) | 16 |
| | LCDR2 (AA) | 17 |
| | LCDR3 (AA) | 18 |
| | Variable heavy region (VH) (AA) | 19 |
| | Variable light region (CL) (AA) | 20 |
| | Heavy chain (AA) | 21 |
| | Light chain (AA) | 22 |
| | Heavy Chain (DNA) | 23 |
| | Light Chain (DNA) | 24 |

| | |
|---|---|
| human oxyntomodulin (53-89) | 25 |
| oxyntomodulin (55-89) | 26 |
| oxyntomodulin (56-89) | 27 |
| human glicentin (21-89) | 28 |
| glicentin span (49-89) | 29 |
| human glucagon (53-81) | 30 |
| glucagon (55-81) | 31 |
| glucagon (56-61) | 32 |
| human glucose-dependent insulinotrophic polypeptide (GIP) | 33 |
| GLP-1 (98-128) | 34 |
| GLP-1 (100-128) | 35 |
| Human proglucagon (1-180) | 36 |

The term “N-terminal region of each of human glucagon and human oxyntomodulin” as used herein refers to the first three amino acid residues in each of those peptides, in which an N-terminal histidine is present.

The term “binds the N-terminal region of each of human glucagon and human oxyntomodulin” as used herein refers to (a) binding to each of human oxyntomodulin (53-59) (SEQ ID NO: 25) and human glucagon (SEQ ID NO: 30), each of which have an N-terminal histidine residue, (b) does not bind to oxyntomodulin (55-89) or oxyntomodulin (56-89), because oxyntomodulin (55-89) and oxyntomodulin (56-89) do not contain the N-terminal histidine residue found in human oxyntomodulin (53-59) and human glucagon (SEQ ID NO: 30), and (c) does not bind to glicentin span (49-89) because although glicentin span (29-69) contains the histidine it is not present with a free amine terminus.

The term “C-terminal region of each of human oxyntomodulin (SEQ ID NO: 25) and human glicentin (SEQ ID NO: 28)” as used herein refers to amino acid residues 30-37 in human oxyntomodulin and amino acid residues 61-69 in human glicentin (SEQ ID NO: 28).

The term “binds the C-terminal region of each of human oxyntomodulin (SEQ ID NO: 25) and human glicentin (SEQ ID NO: 28)” as used herein refers to binding to one or more of amino acid residues 30-37 in human oxyntomodulin and one or more of amino acid residues 61-69 in human glicentin (SEQ ID NO: 28).

The term “N-Terminal Antibody” as used herein refers to the antibody having a heavy chain amino acid sequence of SEQ ID NO: 9 and a light chain amino acid sequence of SEQ ID NO: 10.

The term “C-Terminal Antibody” as used herein refers to the antibody having a heavy chain amino acid sequence of SEQ ID NO: 21 and a light chain amino acid sequence of SEQ ID NO: 22.

The term “polypeptide molecule” as used herein refers to a molecule that comprises a polymer of amino acid residues. In one embodiment, the polypeptide molecule consists of a polymer of amino acid residues.

The term “contacting” as used herein refers to the exposing a substance to another substance. For example, a sample can be exposed to a polypeptide molecule of the invention, for a time and under conditions that permit the polypeptide molecule to bind to human oxyntomodulin present in the sample. Such time and conditions are known to one of skill in the art, and/or can be routinely determined by methods known in the art according to references cited herein.

The term “complex” as used herein refers to the protein-protein interaction between, e.g., a polypeptide molecule and human oxyntomodulin (SEQ ID NO: 25). “First polypeptide molecule-human oxyntomodulin complex” as used herein refers to the protein-protein interaction between a molecule of a polypeptide molecule of the invention and a molecule of human oxyntomodulin (SEQ ID NO: 25). “First polypeptide molecule-human oxyntomodulin-second polypeptide molecule complex” as used herein refers to the concomitant protein-protein interaction between (a) a molecule of a first polypeptide molecule of the invention and a molecule of human oxyntomodulin (SEQ ID NO: 25), and (b) a molecule of a second polypeptide molecule of the invention and the same molecule of human oxyntomodulin molecule (SEQ ID NO: 25).

The term “quantitating the amount of oxyntomodulin” as used herein refers to measuring the amount of oxyntomodulin in a sample, e.g., a liquid sample. Suitable assays are known to those of ordinary skill in the art, such as ELISA.

A polypeptide molecule of the invention can be conjugated to an enzyme and used in an enzyme-linked immunosorbent assay (ELISA). Such assays are described in detail in, for example, Butler (1994) “ELISA” (Chapter 29), In: van Oss, C. J. et al., eds., Immunochemistry, Marcel Dekker, Inc., New York, pp. 759-803. The present polypeptide molecules can also be used in radioimmunoassay and fluorescence-activated cell sorting (FACS) analysis of oxyntomodulin expression.

A particular protein such as human oxyntomodulin can be measured by a variety of immunoassay methods including, e.g., without limitation, competitive and non-competitive assay systems using techniques such as, e.g., without limitation, Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays. For a review of immunological and immunoassay procedures in general, see for example Stites and Terr (eds.), Basic and Clinical Immunology (7th ed.) (1991). Moreover, immunoassays can be performed in many configurations as is known in the art (See for example Maggio (ed.), Enzyme Immunoassay CRC Press, Boca Raton, Florida (1980); Gosling J P, Immunoassays: A Practical Approach (Practical Approach Series), Oxford Univ Press (2000); Diamandis & Christopoulus, Immunoassay, Academic Press (San Diego, CA) (1996). The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org: see Lefranc et al., Nucleic Acids Res. 1999; 27:209-212).

An “antibody fragment” or “antigen-binding fragment” that, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art, for example, the molecular interaction between two molecules, e.g., a polypeptide molecule of the invention and, e.g., human oxyntomodulin (SEQ ID NO: 25). In one embodiment, a polypeptide molecule of the present invention binds specifically to human oxyntomodulin. In another embodiment, “specifically binds” means that a polypeptide molecule of the invention interacts more frequently, more rapidly, with greater duration, with greater affinity, or with some combination of the above to human oxyntomodulin. In another preferred embodiment, “specifically binds” means that a polypeptide molecule of the invention binds to human oxyntomodulin with a K_{D} of about 0.1 mM or less. In another preferred embodiment, “specifically binds” means that a polypeptide molecule of the invention binds to human oxyntomodulin with a K_{D} of about 0.01 mM or less. In another preferred embodiment, “specifically binds” means that a polypeptide molecule of the invention binds to human oxyntomodulin with a K_{D} of about 0.001 mM or less. In another preferred embodiment, “specifically binds” means that a polypeptide molecule of the invention binds to human oxyntomodulin with a K_{D} of about 0.0001 mM or less.

An isolated polynucleotide molecule encoding a HCVR region may be converted to a full-length heavy chain gene by operably linking the HCVR-encoding polynucleotide to another polynucleotide molecule encoding heavy chain constant regions. The sequences of human, as well as other mammalian, heavy chain constant region genes are known in the art. Polynucleotide fragments encompassing these regions may be obtained, e.g., by standard PCR amplification.

An isolated polynucleotide molecule encoding a LCVR region may be converted to a full-length light chain gene by operably linking the LCVR-encoding polynucleotide to another polynucleotide molecule encoding a light chain constant region. The sequences of human, as well as other mammalian, light chain constant region genes are known in the art. Polynucleotide fragments encompassing these regions may be obtained by standard PCR amplification.

The term “sensitivity” as used herein refers to the lowest level of an analyte (in this case, human oxyntomodulin) that can be measured with acceptable accuracy and precision. Sensitivity is reflected for example by the lower limit of quantitation (LLOQ) which is determined, according to the present invention, by taking a sample of oxyntomodulin and diluting it serially until the % CV (coefficient of variation) goes above 20%.

The term “detectably labeled” means that a polypeptide molecule of the present invention, or a complex of oxyntomodulin and polypeptide molecule, hastadedtoit, either covalently or non-covalently, a useful detectable label. In direct conjugate-labeled methods, many different useful labels can be employed including, for example, prosthetic group complexes, chromophores, chromogens (color-producing substrates), dyes, fluorescent compounds, fluorogenic compounds, radioactive isotopes, paramagnetic isotopes, and compounds that can be imaged by positron emission tomography (PET) and magnetic resonance imaging (MRI).

The nucleic acid molecules of the present invention may be expressed in a host cell after the sequences are operably linked to an expression control sequence. The expression vectors are typically replicable in the host organisms either as episomes or as an integral part of the host chromosomal DNA. Commonly, expression vectors contain selection markers, e.g., tetracycline, neomycin, and dihydrofolate reductase, to permit detection of those cells transformed with the desired polynucleotide sequences.

An expression vector containing the nucleic acid sequences of interest (e.g., the nucleic acid sequences encoding one or more of the polypeptide molecules of the invention and expression control sequences) can be transferred into a host cell by known methods, which vary depending on the type of host cells.

A polypeptide molecule of the present invention may be produced in mammalian host cells, non-limiting examples of which include CHO, NS0, HEK293 or COS cells. The host cells may be cultured using techniques known in the art. Polypeptide molecules of the present invention may be expressed and purified essentially as follows. An appropriate host cell, such as HEK 293 or CHO, may be either transiently or stably transfected with an expression system for secreting polypeptide, e.g., an antibody, using an optimal predetermined heavy chain:light chain vector ratio or a single vector system encoding both heavy chain and light chain. Nucleic acid encoding a polypeptide molecule of the present invention may be either transiently or stably transfected with an expression system for secreting polypeptide using one or more DNA molecules encoding for, e.g., an antibody heavy chain and light chain.

Various methods of protein purification may be employed to purify a polypeptide molecule of the present invention and such methods are known in the art and described, for example, in Deutscher, Methods in Enzymology 182: 83-89 (1990) and Scopes, Protein Purfication: Principles and Practice, 3rd Edition, Springer, NY (1994).

For example, the medium may be conveniently applied to a MabSelect column (GE Healthcare Life Sciences), or KappaSelect column (GE Healthcare Life Sciences), that has been equilibrated with a compatible buffer, such as phosphate buffered saline (pH 7.4). The column may be washed to remove nonspecific binding components. The bound polypeptide molecule may be eluted, for example, by pH gradient (such as 20 mM Tris buffer pH 7.0 to 10 mM sodium citrate buffer pH 3.0, or phosphate buffered saline pH 7.4 to 100 mM glycine buffer pH 3.0). Antibody fractions may be detected, such as by UV absorbance or SDS-PAGE, and then may be pooled. Further purification is optional, depending on the intended use. The purified polypeptide molecules may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purified polypeptide molecules may be immediately frozen at −70° C. or may be lyophilized.

The polypeptide molecules disclosed herein are useful for diagnostic, prognostic, and/or patient monitoring procedures, by detecting the level of oxyntomodulin present sum or plasma.

Useful radiolabels, which are detected simply by gamma counter, scintillation counter or autoradiography, include ³H, ¹²⁴I, ¹²⁵I, ¹³¹I, ³⁵S, and ¹⁴C. Radionuclides can be bound to a polypeptide molecule described herein, either directly or indirectly, using a chelating agent, such as DTPA and EDTA. Examples of such radionuclides include ⁹⁹Tc, ¹²³I, ¹²⁵I, ¹³¹I, ¹¹¹In, ⁹⁷Ru, ⁶⁷Cu, ⁶⁷Ga, ⁶⁸Ga, ⁷²As, ⁸⁹Zr, ⁹⁰Y and ²⁰¹Tl.

Other suitable labels are art-known or can be determined by routine experimentation. For example, an antibody, e.g., the T-Terminal Antibody, can be conjugated with, for example, an enzyme. Binding of another antibody to human oxyntomodulin, which itself is also bound to a first the primary antibody, e.g., the C-Terminal Antibody, can then be detected by reaction with a chromogenic substrate of the enzyme under appropriate conditions to yield a detectable signal.

Colorimetric detection can be used, employing chromogenic compounds that have, or result in, chromophores with high extinction coefficients, and which are therefore easily detectable. When later exposed to its substrate under appropriate reaction conditions, the enzyme will react with the substrate to produce a chemical label that can be detected, for example, by spectrophotometric, fluorometric, or visual means.

Enzymes commonly used for this purpose include horseradish peroxidase, alkaline phosphatase, glucose-6-phosphate dehydrogenase, malate dehydrogenase, staphylococcal nuclease, delta-V-steroid isomerase, yeast alcohol dehydrogenase, alpha-glycerophosphate dehydrogenase, triose phosphate isomerase, asparaginase, glucose oxidase, β-galacto-sidase, ribonuclease, urease, catalase, glucoamylase, and acetylcholinesterase.

Non-limiting examples of suitable prosthetic group complexes include, e.g., streptavidin/biotin, avidin/biotin, and neutravidin/biotin. Use of chromogens is preferred because assays employing them can be easily performed in clinical diagnostic laboratories and reviewed by a pathologist with equipment commonly available in these laboratories. Commonly used chromogens include diaminobenzidine (DAB); DAB with enhancement; 3-amino-9-ethyl carbazole (AEC); 4-chloro-1-naphthol (4-CN); Hanker-Yates reagent; alpha-naphthol pyronin; 3,3′,5,5′-tetramethylbenzidine (TMB); Fast Blue BB; Fast Red TR; new fuchsin; BCIP-NBT; tetrazolium; tetranitoblue tetrazolium (TNBT); and immunogold with silver enhancement.

Useful fluorescent labels include umbelliferone, fluorescein, fluorescein isothiocyanate, dichlorotriazinylamine fluorescein, rhodamine, a dansyl group, phycoerythrin, phycocyanin, allophycocyanin, o-phthaldehyde, fluorescamine, and Cy5 (Haugland ((1996) Handbook of Fluorescent Probes and Research Chemicals, Sixth Ed., Molecular Probes, Eugene, OR).

Polypeptide molecules, or polypeptide molecule-oxyntomodulin complexes can also be detectably labeled using fluorescence-emitting metals, such as ¹⁵²Eu⁺, or other members of the lanthanide series, by attaching them using such metal chelating groups as diethylenetriaminepentaacetic acid (DTPA) or ethylenediamine-tetraacetic acid (EDTA).

Polypeptide molecules can also be detectably labeled by coupling them to a phosphorescent or chemiluminescent compound that can then be detected by the phosphorescence or luminescence that arises during the course of a chemical reaction. Examples of useful chemiluminescent compounds include luminol, isoluminol, theromatic acridinium ester, imidazole, acridinium salt, and oxalate ester. Likewise, a bioluminescent compound such as luciferin, luciferase, or aequorin can be used to label the antibody peptides. The presence of a bioluminescent protein is determined by detecting the presence of luminescence.

The present invention also provides articles of manufacture and kits containing compositions useful for quantifying human oxyntomodulin. The article of manufacture may comprise a container with a written label. The container may hold a composition comprising a polypeptide molecule of the present invention, which is either detectably labeled, or unlabeled.

The kit of the present invention can also comprise a container comprising a first and a second polypeptide molecule of the invention. The second polypeptide molecule can be conjugated with an enzyme or other label. A chromogenic substrate of the enzyme can also be included in the kit. The kit may further include other materials desirable from a commercial and user standpoint, including buffers, diluents, filters, needles, syringes, and package inserts with instructions for use in vivo, in vitro, or both.

The following examples are offered for illustrative purposes only, and are not intended to limit the scope of the present invention.

### EXAMPLE 1

### Antibody Binding Studies

Kinetic analysis and binding specificity of the N-Terminal Antibody and the C-Terminal Antibody are determined by surface plasmon resonance using Biacore T100 (GE Healthcare Life Sciences). Goat anti-mouse IgG antibody (Southern Biotech) is immobilized on a Series S CM5 sensor chip and used to capture approximately 1500 response units of anti-oxyntomodulin IgG. Binding to duplicate injections of various concentrations of human oxyntomodulin (SEQ ID NO: 25) (1.1 to 90 nmol/L) diluted in running buffer (HEPES buffered saline containing 3 mmol/L EDTA and 0.05% Tween) and 22.5 nmol/L of either human glicentin span (49-89) (SEQ ID NO: 29) or human glucagon (53-81) (SEQ ID NO: 30) peptides are measured with a flow rate of 30 L/min. Kinetic constants are determined using 1:1 binding model with the T100 Evaluation software.

Using Biacore analysis of the N-Terminal Antibody, the equilibrium dissociation constant is 1.5×10⁻¹⁰ mol/L (Table 2). The N-Terminal Antibody exhibits minimal binding to the glicentin span (48-89) peptide (SEQ ID NO: 29).

TABLE 2
Binding Kinetics of the N-terminal and C-terminal Antibodies

| | ka | kd | KD |
|---|---|---|---|
| Antibody | [(mol/L)⁻¹*s⁻¹]^{a} | (s⁻¹) | (M) |
| N-terminal antibody | 7.2E+06 | 1.0E−03 | 1.5E−10 |
| C-terminal antibody | 1.0E+07 | 8.6E−04 | 8.3E−11 |

Using Biacore analysis of the C-Terminal Antibody, the C-Terminal Antibody dissociation constant is 8.3×10⁻¹¹ mol/L (Table 2). The C-Terminal Antibody exhibits minimal binding to human glucagon (53-81) (SEQ ID NO: 30).

### EXAMPLE 2

### Standard Curve

The optimal antibody pairing for the assay utilizes the C-Terminal Antibody as the capture antibody and N-Terminal Antibody as the detection antibody. A standard curve is prepared with synthetic human oxyntomodulin (SEQ ID NO: 25) that is serially diluted in assay buffer from a starting concentration of 50,000 ng/L. The LLOQ is determined to be 0.4 ng/L based on a 3-SD evaluation from the zero calibrator. The assay demonstrates excellent dynamic range with an upper limit of quantitation (ULOQ) of 2500 ng/L (Table 3). Since oxyntomodulin is a substrate for DDP-4 and this cleavage would remove the N-terminal neo-epitope recognized by the reporting reagent antibody, 6 sets of matched human plasma collected in either P800 or K2 EDTA collection tubes are compared to evaluate the effects from ex-vivo proteolytic degradation after sample collection. An approximate 35-40% reduction in the amount of oxyntomodulin measured with blood collected in the K2 EDTA compared to P800 tubes is observed.

Additional key attributes of the immunoassay are outlined in Table 3.

TABLE 3
Oxyntomodulin Sandwich Immunoassay Characteristics

| | | P800 EDTA |
|---|---|---|
| Parameter | Spike level | plasma |
| Lower Limit of Quantitation (LLOQ) | | 0.388 ng/L |
| Upper Limit of Quantitation (ULOQ) | | 2500 ng/L |
| QC Concentrations (Low, Mid, High) | | 100, 500, and |
| | | 2500 ng/L |
| QC Intra-batch Precision and | Low (CV) | 9.21% |
| Accuracy (n = 9 in one assay) | Mid (CV) | 7.03% |
| | High (CV) | 4.58% |
| QC Inter-batch Precision and | Low (CV) | 10.12% |
| Accuracy (n = 3 in one assay, | Mid (CV) | 3.54% |
| n = 24 total) | High (CV) | 3.03% |
| Dilutional Linearity | High (CV) | 12.0% |
| | Buffer (CV) | 15.0% |
| Recovery | Low (CV) | 113% |
| | Mid (CV) | 102% |
| | High (CV) | 90% |
| Analyte Stability in Thawed Matrix | Low (CV) | 24.0% |
| (room temperature) (QCs stored for | Mid (CV) | 13.0% |
| 4 hours at room temperature.) | High (CV) | 17.0% |
| Analyte Stability in Thawed Matrix | Low (CV) | 14.0% |
| (4° C.) (QCs stored for 24 hours at | Mid (CV) | 0.0% |
| 4° C.) | High (CV) | 8.0% |
| Analyte Stability in Frozen Matrix | Low (CV) | 20.8% |
| (QCs stored for 12 days at −80° | Mid (CV) | 9.47% |
| C. ±10° C.) | High (CV) | 6.80% |
| Reference Range | Mean | 15.13 ng/L |
| (24 individual lots of each matrix; | Standard Dev. | 7.44 |
| n = 1) | | |

QC: Quality Controls

The intra- and inter-batch variability is ≤100/using high (2500 ng/L), mid (500 ng/L) or low (100 ng/L) oxyntomodulin concentrations. The assay demonstrates excellent dilutional linearity with <15% CV after 16-fold dilution of both assay buffer and a human plasma pool spiked with 2500 ng/L of the oxyntomodulin standard. Assay parallelism is observed with the dilution of two different human plasma samples resulting in <20% CV in the calculated values of the analyte. Robustness of the immunoassay using pooled P800 human plasma spiked with 2500, 500, 100, or 0 ng/L oxyntomodulin is demonstrated for a number of parameters. Recoveries ranged from 90-113% for all runs and ≤11% variation in calculated oxyntomodulin values is observed after six freeze-thaw cycles. Finally, the stability of the analyte in P800 collected plasma is demonstrated with <25% CV under the three different temperature conditions outlined in Table 3.

Oxyntomodulin selectivity of the immunoassay is demonstrated by testing binding to a panel of proglucagon or other biologically relevant incretin peptides. Oxyntomodulin immunoassay reactivity to three supra-physiological concentrations ranging from 2500 to 100 ng/L of human glicentin span (49-89) (SEQ ID NO: 29), human glucagon (53-81) (SEQ ID NO: 30), glucose-dependent insulinotrophic polypeptide (GIP) (SEQ ID NO: 33), GLP-1 (98-128) (SEQ ID NO: 34), GLP-1 (100-128) (SEQ ID NO: 35), oxyntomodulin (55-89) (SEQ ID NO: 26), oxyntomodulin (56-89) (SEQ ID NO: 27) is measured. The glicentin span (49-89) (SEQ ID NO: 29) and GIP (SEQ ID NO: 33), peptides are the only analytes that are measured above background levels at the highest concentration. The 500 ng/L concentration of glicentin span (49-89) (SEQ ID NO: 29) also provide a signal slightly above those seen for the buffer blank controls. For comparison and in stark contrast to the glicentin span results, >170,000, >30,000 and >8,000 ECL units are observed with 2500, 500 and 100 ng/L of human oxyntomodulin (53-89) (SEQ ID NO: 25), respectively.

The cross-reactivity of the oxyntomodulin immunoassay is minimal; <0.5% for the glicentin span (49-89) peptide (SEQ ID NO: 29) (which is a surrogate for glicentin cross-reactivity) and 0.12% for GIP peptide (SEQ ID NO: 33). No signal is observed with oxyntomodulin (55-89) peptide (SEQ ID NO: 26) or oxyntomodulin (56-89) peptide (SEQ ID NO: 27), which are N-terminal truncations of human oxyntomodulin of 2 or 3 residues, respectively. No signal is observed when sampling human plasma in which isotype-matched irrelevant antibodies are substituted for either the capture or detection reagent.

### EXAMPLE 3

### Sandwich Assay

Meso Scale Discovery (MSD) Streptavidin Gold Multi-array 96-well plates (Meso Scale Diagnostics) are washed three times with 1× Tris buffered saline (TBS) containing 10 mmol/L Tris pH 7.4, 150 mmol/L NaCl with 1 mL Tween 20/L and blocked with 200 μL of TBS containing 1% (w/v) BSA (Sigma). After one hour, 50 μL of biotin-labeled C-terminal Antibody at a concentration of 1 mg/L is allowed to bind to the plate for an additional hour at room temperature (RT). Oxyntomodulin peptide standards diluted with assay buffer consisting of 50 mmol/L HEPES, pH7.4, 150 mmol/L NaCl, 10 mL/L Triton X-100, 5 mmol/L each EDTA and EGTA, 1% (w/v) BSA, both protease (Roche) and dipeptidyl peptidase-4 (DPP-4) (Millipore) inhibitors, and 100 mg/L Heterophilic Blocking Reagent 1 (Scantibodies) are added to the wells to generate a standard calibration curve. In the same assay buffer, plasma samples are diluted 1:2 and both samples and standards are incubated overnight at 4° C. The plate is washed and 50 μL of 1 ng/mL ruthenium-labeled N-Terminal Antibody is added to the wells and allowed to incubate for 1 hour at RT. Following a final wash step, 150 μL of 2×MSD read buffer is added and a MSD SECTOR Imager 600 (Meso Scale Diagnostics) reader is used to measure ruthenium electrochemiluminescence units (ECL).

Human Plasma Samples. Blood samples from healthy men and women, from 18-65 years of age, are collected into P800 EDTA BD™ Vacutainer (BD Biosciences) collection tubes with informed consent. Samples are stored on ice and spun-down at 4° C. with a bench-top centrifuge at 2000 g for 20 minutes within one hour of collection. Resultant plasma samples are stored at −70° C. prior to analysis of oxyntomodulin levels. K2 and P800 EDTA plasma are also obtained from an additional 19 healthy volunteers under conditions of 10 hour fasting, 5-10 and 90-120 minutes postprandial. The postprandial collection follows a mixed meal challenge consisting of approximately 262 fat calories, 274 carbohydrate calories, and 100 protein calories.

Oxyntomodulin Levels in Normal Human Plasma. The sandwich immunoassay is used to determine the effect of a nutritional challenge on oxyntomodulin plasma levels in healthy individuals. Blood is collected in both P800 and K2 EDTA tubes from 19 volunteers after an overnight fast. Samples are also collected both within a few minutes and within 2 hours after consumption of a standardized mixed meal. Oxyntomodulin values from samples collected with K2 EDTA tubes are lower compared to those collected with P800 collection tubes for all three time points analyzed. The levels of oxyntomodulin increases immediately following feeding. The baseline, early and later time point means for the P800 samples are 11±9 ng/L, 21±9 ng/L and 33±14 ng/L, respectively. Baseline, early and later time point means for the K2 EDTA samples are 87 ng/L, 14±6 ng/L and 23±11 ng/L, respectively.

All data are expressed as means f SEM. MSD Workbench software is used for each of the 4 PL fit calibration curves and interpolation of unknown values. Data are plotted with SigmaPlot version 11.0, and Microsoft Office Excel 2010 or GraphPad Prism 6 is used for data analysis. In each case a p-value of ≤0.05 is considered to indicate statistical significance. Percent cross-reactivities are determined as the ratio of the ECL counts with the peptide of interest to the ECL counts with the reference human oxyntomodulin (53-89) (SEQ ID NO: 25) peptide after subtraction of the buffer blank ECL counts for each concentration tested.

Oxyntomodulin antibody affinities are increased 6 to >200-fold. Intra- and inter-assay CVs are 7-10% and 3-10%, respectively. Spike recovery ranges from 90-113%, linearity is evident up to a 16-fold dilution and glicentin cross-reactivity is 0.5%. Oxyntomodulin levels are lower in K2 EDTA relative to P800 plasma. Postprandial increases in oxyntomodulin occur within minutes and the levels significantly correlated with those obtained using IA-LC-MS.

The oxyntomodulin sandwich immunoassay is appropriately sensitive, selective and is also amendable to high throughput application for the reliable determination of endogenous levels of intact oxyntomodulin from human samples.

The combination of a pair of antibodies that bind to specific human oxyntomodulin N- and C-terminal epitopes, which are present only on the respective proglucagon fragments, facilitates the creation of an immunoassay with sufficient selectivity and sensitivity to determine endogenous levels of human oxyntomodulin.

Selectivity of the immunoassay is initially assessed with other proglucagon derived and incretin peptides of interest. A small signal is observed with the highest spike of glicentin span (49-89) peptide (SEQ ID NO: 29) in the oxyntomodulin sandwich immunoassay; however, this signal is more than 2 orders of magnitude lower than that obtained with the same concentration of human oxyntomodulin (SEQ ID NO: 25). Endogenous glicentin has been reported to increase in a postprandial fashion and peak at 130 pmol/L (Naito H, et al., Regul. Pept. 1999; 79:55-61), and that level is still lower than the high spike concentration (2500 ng/L [560 pmol/L]) used in the studies herein, indicating that endogenous glicentin levels do not impact the human oxyntomodulin sandwich immunoassay herein.

Human oxyntomodulin has a short half-life (minutes) in circulation (Schjoldager B T, et al., Eur. J. Clin. Invest. 1988; 18:499-5), and has been shown to be a substrate for proteolysis by DPP-4 (Yi J, et al., PLoS One 2015; 10:e0134427; Zhu L, et al., J. Biol. Chem. 2003; 278: 22418-23), which removes the first 2 amino-terminal residues. Selectivity of the novel N-terminal neo-epitope antibody disclosed herein (SEQ ID NOS: 9 and 10) is confirmed by the complete lack of reactivity to synthetic oxyntomodulin peptides missing the first 2 or 3 residues (SEQ ID NOS: 26 and 27, respectively). A small but reproducible reduction in the amount of human oxyntomodulin (SEQ ID NO: 25) measured using the sandwich immunoassay herein is observed in sera collected with K2 EDTA, relative to P800 tubes, which confirms that endogenous human oxyntomodulin is processed by DDP-4 or some other related protease that removes N-terminal amino acids, and the sandwich immunoassay disclosed herein selectively measures only the intact human oxyntomodulin (SEQ ID NO: 25) component.

Human K2 EDTA and P800 plasma (500 μL) is spiked with human oxyntomodulin (53-89) SEQ ID NO: 25, oxyntomodulin (55-89) SEQ ID NO: 26 and oxyntomodulin (56-89) SEQ ID: 27 stable-isotope labeled internal standard peptides (CPC Scientific) and diluted with I buffer (25 mmol/L Tris-HCl, 25 mmol/L HEPES, 300 mmol/L NaCl, 0.1% (v/v) octyl β-D-glucopyranoside, pH 7.5). Immunoaffinity enrichment is allowed to proceed overnight at 4° C. following the addition of 2 μg of a biotinylated anti-glucagon antibody (Sloan J H, et al., Clin. Biochem. 2012; 45:1640-4). After incubation, 50 μL of Dynabeads™ MyOne™ streptavidin T1 magnetic beads (ThermoFisher Scientific) are added to the mixture followed by incubation for 30 minutes at RT. The beads are then sequentially washed once with 1 mL of each of the following 3 buffers: radio-immunoprecipitation assay buffer (ThermoFisher Scientific), a buffer consisting of 25 mmol/L Tris-HCl, 25 mmol/L HEPES, 500 mmol/L NaCl, 0.1% (v/v) octyl β-D-glucopyranoside pH 7.5, and deionized water. Bound analytes ae eluted with 50 μL of 0.2% (v/v) formic acid/1× Invitrosol™ (ThermoFisher Scientific)/10% (v/v) acetonitrile. Protein quantification is achieved via high resolution accurate mass LC-MS using a Thermo Scientific Q Exactive mass spectrometer.

Fasting oxyntomodulin levels in healthy human volunteers are found to range from 7-11 ng/L, depending on the collection method employed, and rise within 10 minutes of feeding to levels ranging from 23-33 ng/L at the latest time point collected. These results are consistent with those obtained using the IA-LC-MS assay discussed below, as well as by the LC-MS assay of Lee et al. (Lee A Y, et al., Clin. Chem. 2015; 62: 227-235).

### EXAMPLE 4

### Correlation of Sandwich Assay Results with Immunoassay-LC-MS Assay Results

An oxyntomodulin analytical reference standard is not currently available to gauge the accuracy of the immunoassay. However, immunoassay-LC-MS has the distinct advantage of identifying the molecular forms of an analyte of interest present in a given sample (Bouillon R, et al., Clin. Chem. 2016; 62: 6-8).

The correlation of oxyntomodulin levels obtained with the sandwich immunoassay is examined with those obtained using the immunoassay-LC-MS method of Cox et al. (Cox J M, et al., Bioanalysis 2016; 8:1579-95) on the same set of plasma samples employed in the sandwich assay above. The correlation between the sandwich assay and the immunoassay-LC-MS method is high (Spearman coefficient 0.9236; P<0.0001), and confirms the selectivity of the sandwich immunoassay of the invention.

Moreover, the sandwich immunoassay of the invention enables scalability and high-throughput application, reduction in sample volumes and assay time, and obviates the need for extensive investment in equipment and expertise, relative to the use of immunoassay-LC-MS assays. Use of the sensitive and selective sandwich immunoassay disclosed herein to measure human oxyntomodulin in human samples can also facilitate an improved understanding of oxyntomodulin biology.

Sequences
SEQ ID NO: 1
GYTFTDYAFS
SEQ ID NO: 2
WITTNTGEATYADDFKG
SEQ ID NO: 3
ETEYGDSSWFGH
SEQ ID NO: 4
RASESVDGWGNSFMH
SEQ ID NO: 5
LATYRVA
SEQ ID NO: 6
MQSSEDPYT
SEQ ID NO: 7
QIQLVQSGPELKKPGETVKISCKASGYTFTDYAFSWVKQA
PGKGLKWMGWITTNTGEATYADDFKGRFAFSLETSASTAY
LQISNLKNEDTATYFCARETEYGDSSWFGHWGQGTLVTVS
A
SEQ ID NO: 8
DIVLTQSPASLAVSLGQRATISCRASESVDGWGNSFMHWY
QQKPGQPPKLLIYLATYRVAGIPARFSGSGSRTDFTLTIN
PVEADDVATYYCMQSSEDPYTFGGGTKLEIK
SEQ ID NO: 9
QIQLVQSGPELKKPGETVKISCKASGYTFTDYAFSWVKQA
PGKGLKWMGWITTNTGEATYADDFKGRFAFSLETSASTAY
LQISNLKNEDTATYFCARETEYGDSSWFGHWGQGTLVTVS
AAKTTAPSVYPLAPVCGDTTGSSVTLGCLVKGYFPEPVTL
TWNSGSLSSGVHTFPAVLQSDLYTLSSSVTVTSSTWPSQS
ITCNVAHPASSTKVDKKIEPRGPTIKPCPPCKCPAPNLLG
GPSVFIFPPKIKDVLMISLSPIVTCVVVDVSEDDPDVQIS
WFVNNVEVHTAQTQTHREDYNSTLRVVSALPIQHQDWMSG
KEFKCKVNNKDLPAPIERTISKPKGSVRAPQVYVLPPPEE
EMTKKQVTLTCMVTDFMPEDIYVEWTNNGKTELNYKNTEP
VLDSDGSYFMYSKLR VEKKNWVERNSYSCSVVHEGLHNH
HTTKSFSRTPGK
SEQ ID NO: 10
DIVLTQSPASLAVSLGQRATISCRASESVDGWGNSFMHWY
QQKPGQPPKLLIYLATYRVAGIPARFSGSGSRTDFTLTIN
PVEADDVATYYCMQSSEDPYTFGGGTKLEIKRADAAPTVS
IFPPSSEQLTSGGASVVCFLNNFYPKDINVKWKIDGSERQ
NGVLNSWTDQDSKDSTYSMSSTLTLTKDEYERHNSYTCEA
THKTSTSPIVKSFNRNEC
SEQ ID NO: 11
cagatccagttggtacagtctggacctgagctgaagaagc
ctggagagacagtcaagatctcctgcaaggcttctgggta
taccttcacagactatgcatttagctgggtgaaacaggct
ccaggaaagggtttaaagtggatgggctggataaccacta
acactggagaagccacatatgctgatgacttcaagggacg
gtttgccttctctttggaaacctctgccagcactgcctat
ttgcagatcagcaacctcaaaaatgaggacacggctacat
atttctgtgcaagagagacggaatacggtgatagctcctg
gtttgggcactggggccaagggactctggtcactgtctct
gcagctaaaacaacagccccatcggtctatccgctagccc
ctgtgtgtggagatacaactggctcctcggtgactctagg
atgcctggtcaagggttatttccctgagccagtgaccttg
acctggaactctggcagcctgtccagtggtgtgcacacct
tcccagctgtcctgcagtctgacctctacaccctcagcag
ctcagtgactgtaacgtcgagcacctggcccagccagtcc
atcacctgcaatgtggcccacccggcaagcagcaccaagg
tggacaagaaaattgagcccagagggcccacaatcaagcc
ctgtcctccatgcaaatgcccagcacctaacctcttgggg
gaccatccgtcttcatcttccctccaaagatcaaggatgt
actcatgatctccctgagccccatagtcacatgtgtggtg
gtggatgtgagcgaggatgacccagatgtccagatcagct
ggtttgtgaacaacgtggaagtacacacagctcagacaca
aacccatagagaggattacaacagtactctccgggtggtc
agtgccctccccatccagcaccaggactggatgagtggca
aggagttcaaatgcaaggtcaacaacaaagacctcccagc
gcccatcgagagaaccatctcaaaacccaaagggtcagta
agagctccacaggtatatgtcttgcctccaccagaagaag
agatgactaagaaacaggtcactctgacctgcatggtcac
agacttcatgcctgaagacatttacgtggagtggaccaac
aacgggaaaacagagctaaactacaagaacactgaaccag
tcctggactctgatggttcttacttcatgtacagcaagct
gagagtggaaaagaagaactgggtggaaagaaatagctac
tcctgttcagtggtccacgagggtctgcacaatcaccaca
cgactaagagcttctcccggactccgggtaaa
SEQ ID NO: 12
gacattgtgctgacccaatctccagcttctttggccgtgt
ctctagggcagagggccaccatatcctgcagagccagtga
aagtgttgatggatggggcaatagtttcatgcactggtac
cagcagaaaccaggacagccacccaaactcctcatctatc
tcgctacctatcgcgtagctgggatccctgccaggttcag
tggcagtgggtctaggacagacttcaccctcaccattaat
cctgtggaggctgatgatgttgcaacctattattgtatgc
agagtagtgaagatccgtacacgttcggaggggggaccaa
gctggaaataaaacgggctgatgcggcgcccactgtatcc
atcttcccaccatccagtgagcagttaacatctggaggtg
ctagcgtcgtgtgcttcttgaacaacttctaccccaaaga
catcaatgtcaagtggaagattgatggcagtgaacgacaa
aatggcgtcctgaacagttggactgatcaggacagcaaag
acagcacctacagcatgagcagcaccctcacgttgaccaa
ggacgagtatgaacgacataacagctatacctgtgaggcc
actcacaagacatcaacttcacccattgtcaagagcttca
acaggaatgagtgt
SEQ ID NO: 13
GYNFTNYWLH
SEQ ID NO: 14
ELDPEYGFANYNQKFKG
SEQ ID NO: 15
GFMDY
SEQ ID NO: 16
RSSRSLLDPDGKTYLN
SEQ ID NO: 17
LVSKLDS
SEQ ID NO: 18
WQGTHLPVT
SEQ ID NO: 19
QVQVQQSGAELVMPGTSVKLSCKASGYNFTNYWLHWVKQR
PGQGLEWIGELDPEYGFANYNQKFKGKATLTVDKSSSTAY
MQLSSLTSEDSAVYYCSAGFMDYWGQGTSVTVSS
SEQ ID NO: 20
DVVMTQTPLTLSVNIGQPASISCRSSRSLLDPDGKTYLNW
LLQRPGQSPKRLIYLVSKLDSRVPDRFTGSGSGTDFTLKI
SRVEAEDLGVYYCWQGTHLPVTFGGGTKLEIK
SEQ ID NO: 21
QVQVQQSGAELVMPGTSVKLSCKASGYNFTNYWLHWVKQR
PGQGLEWIGELDPEYGFANYNQKFKGKATLTVDKSSSTAY
MQLSSLTSEDSAVYYCSAGFMDYWGQGTSVTVSSAKTTAP
SVYPLAPVCGDTTGSSVTLGCLVKGYFPEPVTLTWNSGSL
SSGVHTFPAVLQSDLYTLSSSVTVTSSTWPSQSITCNVAH
PASSTKVDKKIEPRGPTIKPCPPCKCPAPNLLGGPSVFIF
PPKIKDVLMISLSPIVTCVVVDVSEDDPDVQISWFVNNVE
VHTAQTQTHREDYNSTLRVVSALPIQHQDWMSGKEFKCKV
NNKDLPAPIERTISKPKGSVRAPQVYVLPPPEEEMTKKQV
TLTCMVTDFMPEDIYVEWTNNGKTELNYKNTEPVLDSDGS
YFMYSKLRVEKKNWVERNSYSCSVVHEGLHNHHTTKSFSR
TPGK
SEQ ID NO: 22
DVVMTQTPLTLSVNIGQPASISCRSSRSLLDPDGKTYLNW
LLQRPGQSPKRLIYLVSKLDSRVPDRFTGSGSGTDFTLKI
SRVEAEDLGVYYCWQGTHLPVTFGGGTKLEIKRADAAPTV
SIFPPSSEQLTSGGASVVCFLNNFYPKDINVKWKIDGSER
QNGVLNSWTDQDSKDSTYSMSSTLTLTKDEYERHNSYTCE
ATHKTSTSPIVKSFNRNEC
SEQ ID NO: 23
caggtccaagtgcagcagtctggggctgagcttgtgatgc
ctgggacttcagtgaagctgtcctgcaaggcctctggcta
taacttcactaattattggttacactgggtgaagcagagg
cctggacaaggccttgagtggatcggagagctcgaccctg
aatatggttttgcgaactacaatcaaaagttcaagggcaa
ggccacattgactgtagacaaatcctccagcacagcctac
atgcagctcagcagcctgacatctgaggactctgcggtct
attactgttctgccgggttcatggactactggggtcaagg
aacctcagtcaccgtctcctcagctaaaacaacagcccca
tcggtctatccgctagcccctgtgtgtggagatacaactg
gctcctcggtgactctaggatgcctggtcaagggttattt
ccctgagccagtgaccttgacctggaactctggcagcctg
tccagtggtgtgcacaccttcccagctgtcctgcagtctg
acctctacaccctcagcagctcagtgactgtaacgtcgag
cacctggcccagccagtccatcacctgcaatgtggcccac
ccggcaagcagcaccaaggtggacaagaaaattgagccca
gagggcccacaatcaagccctgtcctccatgcaaatgccc
agcacctaacctcttgggtggaccatccgtcttcatcttc
cctccaaagatcaaggatgtactcatgatctccctgagcc
ccatagtcacatgtgtggtggtggatgtgagcgaggatga
cccagatgtccagatcagctggtttgtgaacaacgtggaa
gtacacacagctcagacacaaacccatagagaggattaca
acagtactctccgggtggtcagtgccctccccatccagca
ccaggactggatgagtggcaaggagttcaaatgcaaggtc
aacaacaaagacctcccagcgcccatcgagagaaccatct
caaaacccaaagggtcagtaagagctccacaggtatatgt
cttgcctccaccagaagaagagatgactaagaaacaggtc
actctgacctgcatggtcacagacttcatgcctgaagaca
tttacgtggagtggaccaacaacgggaaaacagagctaaa
ctacaagaacactgaaccagtcctggactctgatggttct
tacttcatgtacagcaagctgagagtggaaaagaagaact
gggtggaaagaaatagctactcctgttcagtggtccacga
gggtctgcacaatcaccacacgactaagagcttctcccgg
actccgggtaaa
SEQ ID NO: 24
gatgttgtgatgacccagactccactcactttgtcggtta
acattggacaaccagcctccatctcttgcaggtcaagtcg
gagcctcttagatccagatggaaagacatatttgaattgg
ttgttacagaggccaggccagtctccaaagcgcctaatct
atctggtgtctaaactggactctagagtccctgacaggtt
cactggcagtggatcagggacagatttcacactgaaaatc
agcagagtggaggctgaggatttgggagtttattattgct
ggcaaggtacacatcttcctgtaacgttcggtggaggcac
caagctggaaatcaaacgggctgatgcggcgcccactgta
tccatcttcccaccatccagtgagcagttaacatctggag
gtgctagcgtcgtgtgcttcttgaacaacttctaccccaa
agacatcaatgtcaagtggaagattgatggcagtgaacga
caaaatggcgtcctgaacagttggactgatcaggacagca
aagacagcacctacagcatgagcagcaccctcacgttgac
caaggacgagtatgaacgacataacagctatacctgtgag
gccactcacaagacatcaacttcacccattgtcaagagct
tcaacaggaatgagtgt
SEQ ID NO: 25
HSQGTFTSDYSKYLDSRRAQDFVQWLMNTKRNRNNIA
SEQ ID NO: 26
QGTFTSDYSKYLDSRRAQDFVQWLMNTKRNRNNIA
SEQ ID NO: 27
GTFTSDYSKYLDSRRAQDFVQWLMNTKRNRNNIA
SEQ ID NO: 28
RSLQDTEEKSRSFSASQADPLSDPDQMNEDKRHSQGTFTS
DYSKYLDSRRAQDFVQWLMNTKRNRNNIA
SEQ ID NO: 29
EDKRHSQGTFTSDYSKYLDSRRAQDFVQWLMNTKRNRNNIA
SEQ ID NO: 30
HSQGTFTSDYSKYLDSRRAQDFVQWLMNT
SEQ ID NO: 31
QGTFTSDYSKYLDSRRAQDFVQWLMNT
SEQ ID NO: 32
GTFTSDYSKYLDSRRAQDFVQWLMNT
SEQ ID NO: 33
YAEGTFISDYSIAMDKIHQQDFVNWLLAQKGKKNDWKHNITQ
SEQ ID NO: 34
HAEGTFTSDVSSYLEGQAAKEFIAWLVKGRG
SEQ ID NO: 35
EGTFTSDVSSYLEGQAAKEFIAWLVKGRG
SEQ ID NO: 36
MKSIYFVAGLFVMLVQGSWQRSLQDTEEKSRSFSASQADP
LSDPDQMNEDKRHSQGTFTSDYSKYLDSRRAQDFVQWLMN
TKRNRNNIAKRHDEFERHAEGTFTSDVSSYLEGQAAKEFI
AWLVKGRGRRDFPEEVAIVEELGRRHADGSFSDEMNTILD
NLAARDFINWLIQTKITDRK

We claim:

1. A polypeptide molecule selected from the group consisting of
(a) a polypeptide molecule that binds the N-terminal region of each of human oxyntomodulin (SEQ ID NO: 25) and human glucagon (SEQ ID NO: 30), wherein the polypeptide molecule comprises the complementarity determining regions (CDRs) set forth in SEQ ID NOS: 1-6 and
(b) a polypeptide molecule that binds the C-terminal region in each of human oxyntomodulin (SEO ID NO: 25) and human glicentin (SEO ID NO: 28), wherein the polypeptide molecule comprises the complementarity determining regions (CDRs) set forth in SEQ ID NOS: 13-18.

2. The polypeptide molecule of claim 1, wherein the polypeptide molecule is an antibody.

3. The polypeptide molecule of claim 1, wherein the polypeptide molecule is an scFv or a Fab.

4. The polypeptide molecule of claim 1(a), wherein the polypeptide molecule is an antibody comprising a heavy chain variable region (VH) comprising SEQ ID NO: 7 and a light chain variable region (VL) comprising SEQ ID NO: 8.

5. The polypeptide molecule of claim 4, wherein the polypeptide molecule is an antibody comprising a heavy chain comprising SEQ ID NO: 9 and a light chain comprising SEQ ID NO: 10.

6. The polypeptide molecule of claim 5, wherein the polypeptide molecule is an antibody comprising a heavy chain consisting of SEQ ID NO: 9 and a light chain consisting of SEQ ID NO: 10.

7. A nucleic acid molecule selected from the group consisting of
(a) a nucleic acid molecule comprising one or both of a first nucleic acid sequence encoding SEQ ID NO: 9 and a second nucleic acid sequence encoding SEQ ID NO: 10 and
(b) a nucleic acid molecule comprising one or both of a first nucleic acid sequence encoding SEQ ID NO: 21 and a second nucleic acid sequence encoding SEQ ID NO: 22.

8. The nucleic acid molecule of claim 7(a), wherein the first nucleic acid sequence encoding SEQ ID NO: 9 comprises SEQ ID NO: 11, and wherein the second nucleic acid sequence encoding SEO ID NO: 10 comprises SEQ ID NO: 12.

9. A vector selected from the group consisting of
(a) a vector comprising one or both of a first nucleic acid sequence encoding SEQ ID NO: 9 and a second nucleic acid sequence encoding SEQ ID NO: 10 and
(b) a vector comprising one or both of a first nucleic acid sequence encoding SEQ ID NO: 21 and a second nucleic acid sequence encoding SEQ ID NO: 22.

10. A composition comprising the vector of claim 9.

11. A cell comprising the vector of claim 9.

12. The cell of claim 11, wherein the cell is a mammalian cell.

13. A process of producing a polypeptide molecule, comprising culturing a cell comprising (a) one or both of a first nucleic acid sequence encoding SEQ ID NO: 9 and a second nucleic acid sequence encoding SEQ ID NO: 10 or (b) one or both of a first nucleic acid sequence encoding SEO ID NO: 21 and a second nucleic acid sequence encoding SEQ ID NO: 22, under conditions such that the polypeptide molecule is expressed, and recovering the expressed polypeptide molecule from the culture medium.

14. A polypeptide molecule produced by the process of claim 13.

15. A composition comprising the polypeptide molecule of claim 14.

16. (canceled)

17. (canceled)

18. (canceled)

19. The polypeptide molecule of claim 1(b), wherein the polypeptide molecule is an antibody comprising a VH comprising SEQ ID NO: 19 and a VL comprising SEQ ID NO: 20.

20. The polypeptide molecule of claim 19, wherein the polypeptide molecule is an antibody comprising a heavy chain comprising SEQ ID NO: 21 and a light chain comprising SEQ ID NO: 22.

21. The polypeptide molecule of claim 20, wherein the polypeptide molecule is an antibody comprising a heavy chain consisting of a SEQ ID NO:21 and a light chain consisting of SEQ ID NO: 22.

22. (canceled)

23. The nucleic acid molecule of claim 7(b), wherein the first nucleic acid sequence encoding SEQ ID NO: 21 comprises SEQ ID NO: 23, and wherein the second nucleic acid sequence encoding SEQ ID NO: 22 comprises SEQ ID NO: 24.

24. (canceled)

25. (canceled)

26. (canceled)

27. (canceled)

28. (canceled)

29. (canceled)

30. (canceled)

31. A composition comprising the polypeptide of claim 1.

32. A method of determining the concentration of human oxyntomodulin (SEQ ID NO: 25) in a liquid sample, comprising:
(a) contacting a liquid sample comprising human oxyntomodulin with a first polypeptide molecule that binds the C-terminal region in each of human oxyntomodulin (SEQ ID NO: 25) and human glicentin (SEQ ID NO: 28), wherein the first polypeptide molecule comprises the CDRs of SEQ ID NOS: 13-18, thereby forming a first polypeptide molecule-human oxyntomodulin complex;
(b) contacting the first polypeptide molecule-human oxyntomodulin complex with a second polypeptide molecule that binds the N-terminal region of each of human oxyntomodulin (SEQ ID NO: 25) and human glucagon (SEQ ID NO: 30), wherein the second polypeptide molecule comprises the CDRs of SEQ ID NOS: 1-6, thereby forming a first polypeptide molecule-human oxyntomodulin-second polypeptide molecule complex; and
(c) quantitating the amount of oxyntomodulin in the first polypeptide molecule-human oxyntomodulin-second polypeptide molecule complex by comparison against a standard curve of known amounts of human oxyntomodulin (SEQ ID NO: 25).

33. The method of claim 32, wherein the liquid sample is serum or plasma.

34. The method of claim 32, wherein the first polypeptide molecule is an antibody and the second polypeptide molecule is an antibody.

35. The method of claim 32, wherein the first polypeptide molecule is an antibody comprising a VH comprising SEQ ID NO: 19 and a VL comprising SEQ ID NO: 20, and the second polypeptide molecule is an antibody comprising a VH comprising SEQ ID NO: 7 and a VL comprising SEQ ID NO: 8.

36. The method of claim 32, wherein the first polypeptide molecule is an antibody comprising a heavy chain comprising SEQ ID NO: 21 and comprising a light chain comprising SEQ ID NO: 22, and the second polypeptide molecule is an antibody comprising a heavy chain comprising SEQ ID NO: 9 and a light chain comprising SEQ ID NO: 10.

37. The method of claim 32, wherein the first polypeptide molecule is an antibody comprising a heavy chain consisting of SEQ ID NO: 21 and a light chain consisting of SEQ ID NO: 22, and the second polypeptide molecule is an antibody comprising a heavy chain consisting of SEQ ID NO: 9 and a light chain consisting of SEQ ID NO: 10.

38. (canceled)

39. A method of predicting if a subject is at risk of developing type 2 diabetes, comprising determining the amount of oxyntomodulin in a serum sample or a plasma sample from a subject by:
(a) contacting serum sample or a plasma sample comprising human oxyntomodulin with a first polypeptide molecule that binds the C-terminal region of each of human oxyntomodulin (SEQ ID NO: 25) and human glicentin (SEQ ID NO: 28), wherein the first polypeptide molecule comprises SEQ ID NOS: 13-18, thereby forming a first polypeptide molecule-human oxyntomodulin complex;
(b) contacting the first polypeptide molecule-human oxyntomodulin complex with a second polypeptide molecule the N-terminal region of each of human oxyntomodulin (SEQ ID NO: 25) and human glucagon (SEQ ID NO: 30), wherein the second polypeptide molecule comprises SEQ ID NOS: 1-6, thereby forming a first polypeptide molecule-human oxyntomodulin-second polypeptide molecule complex; and
(c) quantitating the amount of oxyntomodulin in the first polypeptide molecule-human oxyntomodulin-second polypeptide molecule complex by comparison against a standard curve of known amounts of human oxyntomodulin (SEQ ID NO: 25).

40. The method of claim 39, wherein the subject has been diagnosed with acute pancreatitis.

41. The method of claim 39, wherein the first polypeptide molecule is an antibody and the second polypeptide molecule is an antibody.

42. The method of claim 39 wherein the first polypeptide molecule is an antibody comprising a variable heavy region comprising SEQ ID NO: 19 and a variable light region comprising SEQ ID NO: 20, and the second polypeptide molecule is an antibody comprising a variable heavy region comprising SEQ ID NO: 7 and a variable light region comprising SEQ ID NO: 8.

43. The method of claim 39, wherein the first polypeptide molecule is an antibody comprising a heavy chain comprising SEQ ID NO: 21 and comprising a light chain comprising SEQ ID NO: 22, and the second polypeptide molecule is an antibody comprising a heavy chain comprising SEQ ID NO: 9 and a light chain comprising SEQ ID NO: 10.

44. The method of claim 39, wherein the first polypeptide molecule is an antibody comprising a heavy chain consisting of SEQ ID NO: 21 and comprising a light chain consisting of SEQ ID NO: 22, and the second polypeptide molecule is an antibody comprising a heavy chain consisting of SEQ ID NO: 9 and a light chain consisting of SEQ ID NO: 10.


SEQUENCE LISTING (36 sequences)

SEQ ID NO: 1 - protein, 10 aa, Artificial Sequence
  Synthetic Construct
GYTFTDYAFS

SEQ ID NO: 2 - protein, 17 aa, Artificial Sequence
  Synthetic Construct
WITTNTGEATYADDFKG

SEQ ID NO: 3 - protein, 12 aa, Artificial Sequence
  Synthetic Construct
ETEYGDSSWFGH

SEQ ID NO: 4 - protein, 15 aa, Artificial Sequence
  Synthetic Construct
RASESVDGWGNSFMH

SEQ ID NO: 5 - protein, 7 aa, Artificial Sequence
  Synthetic Construct
LATYRVA

SEQ ID NO: 6 - protein, 9 aa, Artificial Sequence
  Synthetic Construct
MQSSEDPYT

SEQ ID NO: 7 - protein, 121 aa, Artificial Sequence
  Synthetic Construct
QIQLVQSGPELKKPGETVKISCKASGYTFTDYAFSWVKQAPGKGLKWMGWITTNTGEATYADDFKGRFAFSLETSASTAYLQISNLKNEDTATYFCARETEYGDSSWFGHWGQGTLVTVSA

SEQ ID NO: 8 - protein, 111 aa, Artificial Sequence
  Synthetic Construct
DIVLTQSPASLAVSLGQRATISCRASESVDGWGNSFMHWYQQKPGQPPKLLIYLATYRVAGIPARFSGSGSRTDFTLTINPVEADDVATYYCMQSSEDPYTFGGGTKLEIK

SEQ ID NO: 9 - protein, 451 aa, Artificial Sequence
  Synthetic Construct
QIQLVQSGPELKKPGETVKISCKASGYTFTDYAFSWVKQAPGKGLKWMGWITTNTGEATYADDFKGRFAFSLETSASTAYLQISNLKNEDTATYFCARETEYGDSSWFGHWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 218 aa, Artificial Sequence
  Synthetic Construct
DIVLTQSPASLAVSLGQRATISCRASESVDGWGNSFMHWYQQKPGQPPKLLIYLATYRVAGIPARFSGSGSRTDFTLTINPVEADDVATYYCMQSSEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - DNA, 1353 bp, Artificial Sequence
  Synthetic Construct
cagatccagttggtacagtctggacctgagctgaagaagcctggagagacagtcaagatctcctgcaaggcttctgggtataccttcacagactatgcatttagctgggtgaaacaggctccaggaaagggtttaaagtggatgggctggataaccactaacactggagaagccacatatgctgatgacttcaagggacggtttgccttctctttggaaacctctgccagcactgcctatttgcagatcagcaacctcaaaaatgaggacacggctacatatttctgtgcaagagagacggaatacggtgatagctcctggtttgggcactggggccaagggactctggtcactgtctctgcagctaaaacaacagccccatcggtctatccgctagcccctgtgtgtggagatacaactggctcctcggtgactctaggatgcctggtcaagggttatttccctgagccagtgaccttgacctggaactctggcagcctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 12 - DNA, 654 bp, Artificial Sequence
  Synthetic Construct
gacattgtgctgacccaatctccagcttctttggccgtgtctctagggcagagggccaccatatcctgcagagccagtgaaagtgttgatggatggggcaatagtttcatgcactggtaccagcagaaaccaggacagccacccaaactcctcatctatctcgctacctatcgcgtagctgggatccctgccaggttcagtggcagtgggtctaggacagacttcaccctcaccattaatcctgtggaggctgatgatgttgcaacctattattgtatgcagagtagtgaagatccgtacacgttcggaggggggaccaagctggaaataaaacgggctgatgcggcgcccactgtatccatcttcccaccatccagtgagcagttaacatctggaggtgctagcgtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 13 - protein, 10 aa, Artificial Sequence
  Synthetic Construct
GYNFTNYWLH

SEQ ID NO: 14 - protein, 17 aa, Artificial Sequence
  Synthetic Construct
ELDPEYGFANYNQKFKG

SEQ ID NO: 15 - protein, 5 aa, Artificial Sequence
  Synthetic Construct
GFMDY

SEQ ID NO: 16 - protein, 16 aa, Artificial Sequence
  Synthetic Construct
RSSRSLLDPDGKTYLN

SEQ ID NO: 17 - protein, 7 aa, Artificial Sequence
  Synthetic Construct
LVSKLDS

SEQ ID NO: 18 - protein, 9 aa, Artificial Sequence
  Synthetic Construct
WQGTHLPVT

SEQ ID NO: 19 - protein, 114 aa, Artificial Sequence
  Synthetic Construct
QVQVQQSGAELVMPGTSVKLSCKASGYNFTNYWLHWVKQRPGQGLEWIGELDPEYGFANYNQKFKGKATLTVDKSSSTAYMQLSSLTSEDSAVYYCSAGFMDYWGQGTSVTVSS

SEQ ID NO: 20 - protein, 112 aa, Artificial Sequence
  Synthetic Construct
DVVMTQTPLTLSVNIGQPASISCRSSRSLLDPDGKTYLNWLLQRPGQSPKRLIYLVSKLDSRVPDRFTGSGSGTDFTLKISRVEAEDLGVYYCWQGTHLPVTFGGGTKLEIK

SEQ ID NO: 21 - protein, 444 aa, Artificial Sequence
  Synthetic Construct
QVQVQQSGAELVMPGTSVKLSCKASGYNFTNYWLHWVKQRPGQGLEWIGELDPEYGFANYNQKFKGKATLTVDKSSSTAYMQLSSLTSEDSAVYYCSAGFMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 219 aa, Artificial Sequence
  Synthetic Construct
DVVMTQTPLTLSVNIGQPASISCRSSRSLLDPDGKTYLNWLLQRPGQSPKRLIYLVSKLDSRVPDRFTGSGSGTDFTLKISRVEAEDLGVYYCWQGTHLPVTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - DNA, 1332 bp, Artificial Sequence
  Synthetic Construct
caggtccaagtgcagcagtctggggctgagcttgtgatgcctgggacttcagtgaagctgtcctgcaaggcctctggctataacttcactaattattggttacactgggtgaagcagaggcctggacaaggccttgagtggatcggagagctcgaccctgaatatggttttgcgaactacaatcaaaagttcaagggcaaggccacattgactgtagacaaatcctccagcacagcctacatgcagctcagcagcctgacatctgaggactctgcggtctattactgttctgccgggttcatggactactggggtcaaggaacctcagtcaccgtctcctcagctaaaacaacagccccatcggtctatccgctagcccctgtgtgtggagatacaactggctcctcggtgactctaggatgcctggtcaagggttatttccctgagccagtgaccttgacctggaactctggcagcctgtccagtggtgtgcacaccttcccagctgtcctgcagtctgacctctacaccctcagcagctcagtgactgtaacgtcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 24 - DNA, 657 bp, Artificial Sequence
  Synthetic Construct
gatgttgtgatgacccagactccactcactttgtcggttaacattggacaaccagcctccatctcttgcaggtcaagtcggagcctcttagatccagatggaaagacatatttgaattggttgttacagaggccaggccagtctccaaagcgcctaatctatctggtgtctaaactggactctagagtccctgacaggttcactggcagtggatcagggacagatttcacactgaaaatcagcagagtggaggctgaggatttgggagtttattattgctggcaaggtacacatcttcctgtaacgttcggtggaggcaccaagctggaaatcaaacgggctgatgcggcgcccactgtatccatcttcccaccatccagtgagcagttaacatctggaggtgctagcgtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 25 - protein, 37 aa, Homo sapiens
HSQGTFTSDYSKYLDSRRAQDFVQWLMNTKRNRNNIA

SEQ ID NO: 26 - protein, 35 aa, Homo sapiens
QGTFTSDYSKYLDSRRAQDFVQWLMNTKRNRNNIA

SEQ ID NO: 27 - protein, 34 aa, Homo sapiens
GTFTSDYSKYLDSRRAQDFVQWLMNTKRNRNNIA

SEQ ID NO: 28 - protein, 69 aa, Homo sapiens
RSLQDTEEKSRSFSASQADPLSDPDQMNEDKRHSQGTFTSDYSKYLDSRRAQDFVQWLMNTKRNRNNIA

SEQ ID NO: 29 - protein, 41 aa, Homo sapiens
EDKRHSQGTFTSDYSKYLDSRRAQDFVQWLMNTKRNRNNIA

SEQ ID NO: 30 - protein, 29 aa, Homo sapiens
HSQGTFTSDYSKYLDSRRAQDFVQWLMNT

SEQ ID NO: 31 - protein, 27 aa, Homo sapiens
QGTFTSDYSKYLDSRRAQDFVQWLMNT

SEQ ID NO: 32 - protein, 26 aa, Homo sapiens
GTFTSDYSKYLDSRRAQDFVQWLMNT

SEQ ID NO: 33 - protein, 42 aa, Homo sapiens
YAEGTFISDYSIAMDKIHQQDFVNWLLAQKGKKNDWKHNITQ

SEQ ID NO: 34 - protein, 31 aa, Homo sapiens
HAEGTFTSDVSSYLEGQAAKEFIAWLVKGRG

SEQ ID NO: 35 - protein, 29 aa, Homo sapiens
EGTFTSDVSSYLEGQAAKEFIAWLVKGRG

SEQ ID NO: 36 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK